(19) United States
(12) Patent Application Publication
(10) Pub. No.: US 2013/0150928 A1
(43) Pub. Date: Jun. 13, 2013

(54) Litter with Integrated Power Supply

(71) Applicant: NORTH AMERICAN RESCUE, LLC, Greer, SC (US)

(72) Inventors: Joanne S. Walter, Blue Ridge, GA (US); Darrel Saunders, Simpsonville, SC (US)

(73) Assignee: NORTH AMERICAN RESCUE, LLC, Greer, SC (US)

(21) Appl. No.: 13/707,063

(22) Filed: Dec. 6, 2012

Related U.S. Application Data
(60) Provisional application No. 61/568,560, filed on Dec. 8, 2011.

(51) Int. Cl. A61F 7/08

(52) U.S. Cl. CPC A61F 7/08
USPC 607/96

(57) ABSTRACT

This invention is a portable collapsible field litter comprising: a frame for supporting a tent and having a plurality of frame sections hingeably attached wherein the frame and the tent have a collapsed position for storage and transportation and an extended position for supporting an individual; a cavity defined internal to one of the frame sections for receiving a power cell within the frame section; a medical device connector attached to the frame and in electrical communications with the power cell through a plurality of contacts disposed in the cavity providing an electrical connection between the power cell and the medical device connector; a heated wrap electrically connected to the medical device connector so that the heated wrap is heated using power delivered from the power cell, the heated wrap is disposed around the individual supported by the tent; and, a heat loss fabric disposed around the individual and the heated wrap to reduce heat radiating away from the individual to assist in preventing hypothermia.

### CLAIM OF PRIORITY

This application claims priority on U.S. Provisional Patent Application Ser. No. 61/568,560 filed Dec. 8, 2012.

### FIELD OF THE INVENTION

This invention is directed to a litter for transporting injured persons, and more specifically, a litter including a power supply for powering medical device accessories.

### BACKGROUND OF THE INVENTION

In the treatment of injured persons, especially in the field, there are powered medical accessories which can reduce or eliminate some life-threatening conditions. Such medical devices include patient monitoring devices, wound illumination devices, heating and cooling devices and the like. For example, hypothermia is a condition which, if not properly treated, can be lethal. Hypothermia has been described as one of the most preventable causes of combat or trauma related death. This is true because of the treatment of hypothermia is known and does not require expensive or complex equipment.

Equipment used to help prevent hypothermia includes passive equipment such as the HPMK® sold by North American Rescue, LLC as well as active powered equipment such as the ARC° tc Thermal Wrap. The HPMK® is a heat loss prevention blanket used to prevent hypothermia. Treatment includes heating IV fluid administered to the patient as well as warmed, humidified air delivered to the respiratory system of the patient. Both heating IV fluid and air are from powered units.

One method for powering such units is the Standard Military BA-5590 battery. The BA-5590 is one of the most common batteries in the military and provides 15 volts of power at 7.5 amps. While commonly known as a 12-volt battery, in actuality it contains two strings of five 3.0-volt lithium sulfur dioxide cells. The connector is a female 6 pin floating configuration. Pins 1 and 2 are ground and 4 and 5 are positive, allowing the BA-5590 to be wired in parallel (15 V) or series (30 V). The typical weight is around 2.2 pounds.

When treating hypothermia, the ARC° tc is a heated vest that can deliver 104° F. to a patient within 90 seconds and will run for at least five hours when powered by the BA-5590. In one configuration, the vest is placed on the patient, powered and the patient and ARC° tc Thermao Wrap are placed into a passive HPMK®. The patient, with ARC° tc Thermal Wrap and HPMK®, can be placed on a litter and transported. However, there is no efficient or secure method to transport the BA-5590 with the litter. Traditionally, it was simply placed on or beside the patient on the litter. This caused the BA-5590 to be prone to falling off the litter and disconnecting from the ARC° tc or pulling the ARC° tc out of place or off the patient. This disadvantage exists for each medical device or other powered item used in connection with a patient being transported on a litter.

Therefore, it is an object of the present invention to provide a lightweight power unit which is not prone to falling off a litter.

It is another object of the present invention to provide a power source integrated into a litter with sufficient power to operate medical treatment devices.

### SUMMARY OF THE INVENTION

The objects of the present invention are accomplished by providing a portable collapsible field litter comprising: a frame for supporting a tent and having a plurality of frame sections hingeably attached wherein the frame and the tent have a collapsed position for storage and transportation and an extended position for supporting an individual; a cavity defined internal to one of the frame sections for receiving a power cell within the frame section; a medical device connector attached to the frame and in electrical communications with the power cell through a plurality of contacts disposed in the cavity providing an electrical connection between the power cell and the medical device connector; a heated wrap electrically connected to the medical device connector so that the heated wrap is heated using power delivered from the power cell, the heated wrap is disposed around the individual supported by the tent; and, a heat loss fabric disposed around the individual and the heated wrap to reduce heat radiating away from the individual to assist in preventing hyperthermia.

The medical device connector can be of the BA-5590 type connector. The heated wrap can be a medical device taken from the group consisting of: ARCtc Cocoon BA, ARCtc HPMK BA and ARCtc ThermalWrap BA. Each of these devices are available from the vendor North American Rescue, LLC of Greer, S.C. and correspond to part numbers: 80-0253; 8-0239; 80-0252 and 80-0224.

Included in the invention can be an indicator electrically connected to the power cell wherein the indicator indicates status taken from the group consisting of: power level, medical device connection, remaining power, and any combination thereof. The indicator can indicate the status of various aspects of the invention including power level, medical device connection, remaining power, and any combination thereof. The medical devices that can be powered by the present invention can include patient monitoring devices, wound illumination devices, heating and cooling devices and the like.

The invention can also include a power switch electrically in-line between the power cell and the medical device connector for allowing power from the power cell to be interrupted or delivered to the medical device connector when the power switch is actuated.

The cavity receiving the power cell can be defined in a proximal end of a handle section of the frame. An end cap for securing the power cell in the cavity in the handle section can be included. A sleeve for receiving the power cell whereas the sleeve containing the power cells is received in the cavity, the sleeve having an electrical connection between the power cell and the electrical contacts allowing power to be transmitted from the power cell to the electrical contacts can be included. The invention can also include a first end cap having a medical device connector removable attached to a proximal end of the sleeve and a second end cap having internal electrical contacts removable attached to a distal end of the sleeve. The power cell can be affixed within the cavity and rechargeable through the medical device connector.

### DESCRIPTION OF THE DRAWINGS

The description of the invention is better understood by referring to the following figures:

FIGS. 1A and 1B are schematics of the invention;

FIGS. 1C and 1D are a perspective views of aspects of the present invention;

FIGS. 2A through 2D are perspective views of aspects of the invention; and,

FIG. 3 is a schematic of the invention.

### DETAILED DESCRIPTION OF THE DRAWINGS

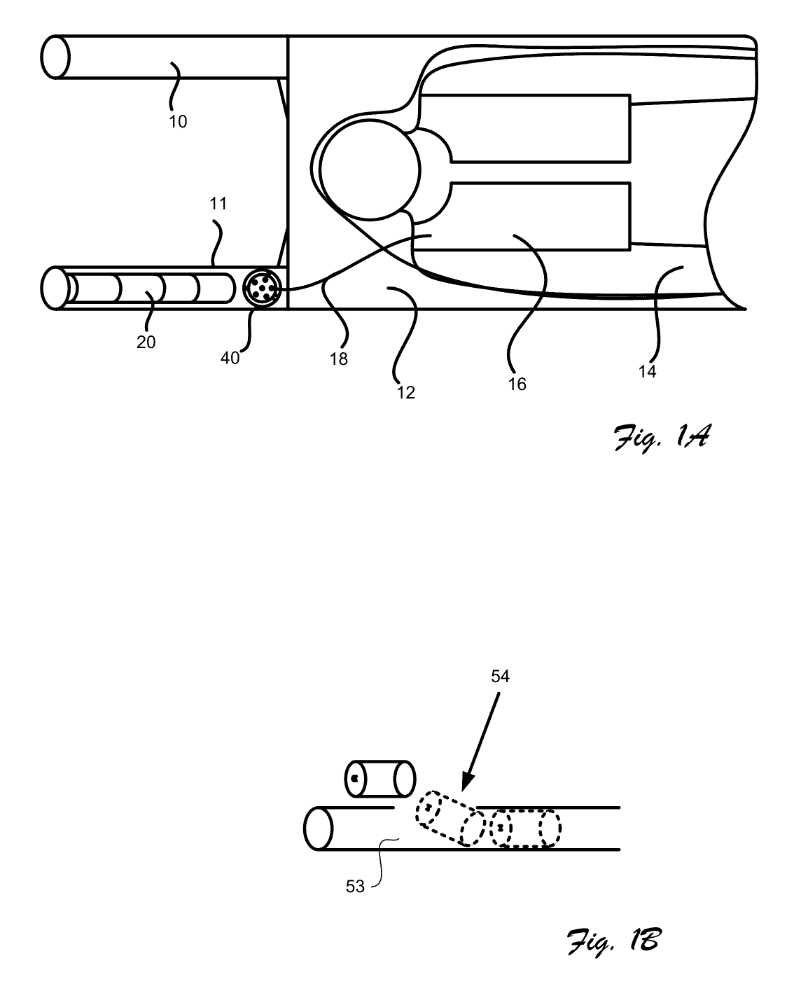

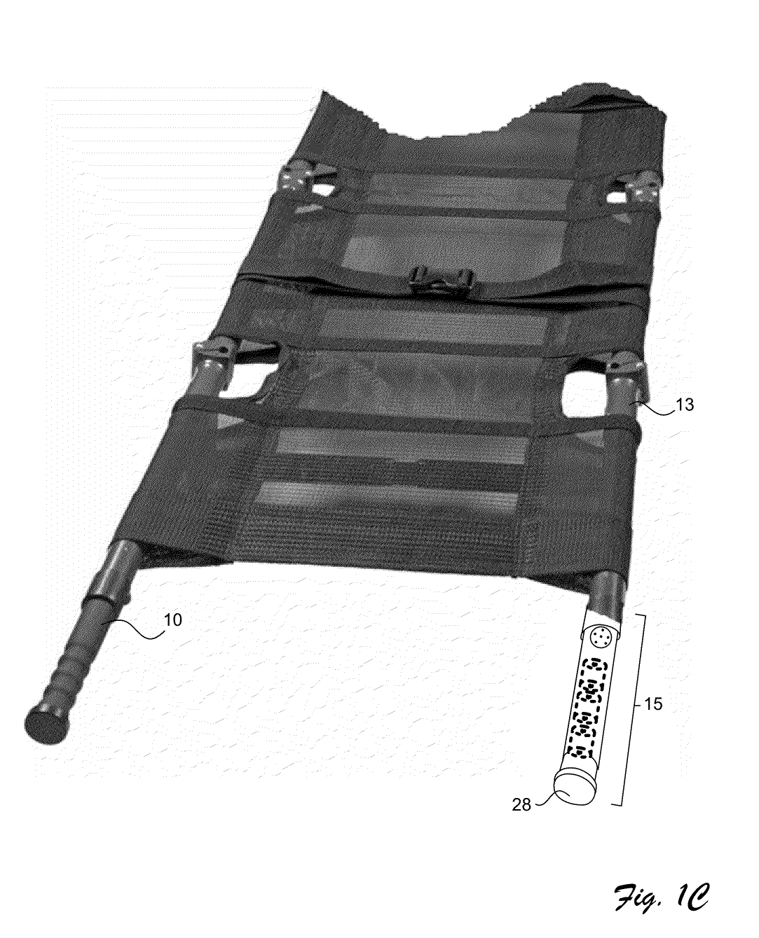

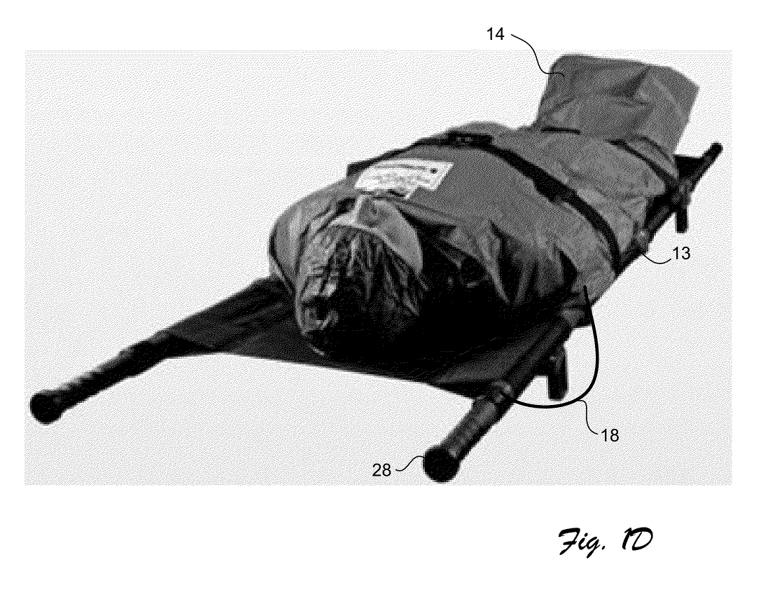

Referring to FIG. 1, a litter 10 is shown including a tent 12 which supports the patient. The patient can be covered by a heat loss prevention fabric 14 and a powered medical device such a powered heated wrap 16. The wrap should be positioned to cover the chest area of the patient evenly. Traditional power sources can pull on the power cord to eh heated vest and cause the vest to be misaligned. It is a disadvantage and dangerous for the heating vest to fall out of position on the patient.

The heating vest can include a power cord 18 which is in electrical connection to a power cell 20. The power cell can be received into a cavity defined in the frame or frame section internal to the frame section. The powered medical device can connect to the power cell through medical device connector 40. In one embodiment, the medical connector device is molded into a frame section 11. In one embodiment, the frame section is a handle section disposed at the ends of the frame. The handle section can include extendable handle grips.

The power supply can be any DC or AC power source. In one embodiment, the power cell includes a plurality of cells in series to produce 12 or more volts. In one embodiment, the cells are CR123-type batteries. The power cells can be replaceable, rechargeable, or both. In one embodiment, the power cell is affixed to the internal cavity of the frame section and rechargeable.

Referring to FIG. 1B, the power cell can be received into a cavity 53 through an opening 54 defined in the outer surface of the frame or frame section. Electrical contacts can be included in the cavity providing an electrical connection between the power cell and the medical device connector. A cover can be included to cover opening 54 allowing access to the cavity to insert and remove the power cell or power cells.

In another embodiment, the sleeve can have a slot or rail which can correspond to a rail or slot in the opening of the frame or frame section so that the sleeve can only be received by the frame section in a specific orientation. The connectors on the sleeve can be points or strips so that the electrical connection between the sleeve and the contacts in the cavity of the frame section can be made independent of the sleeve's orientation within the frame section.

Referring to FIG. 1 C, the frame is shown with power cells 20 received into a cavity in a handle section 13. In this embodiment, handle section includes a grip 15 that can be retracted into the handle section. The power cells can therefore also be retracted into the handle section when the handle grip is retracted. In one embodiment, the handle grip can be removed to allow the power cell to be inserted into a cavity defined in the handle grip. In another embodiment, end cap 28 can be removed to allow the power cell to be inserted or removed from the cavity. FIG. 1D shows the invention with an individual supported by the tent and surrounded with a heat loss material. The power cord for the powered medical device is shown as 18.

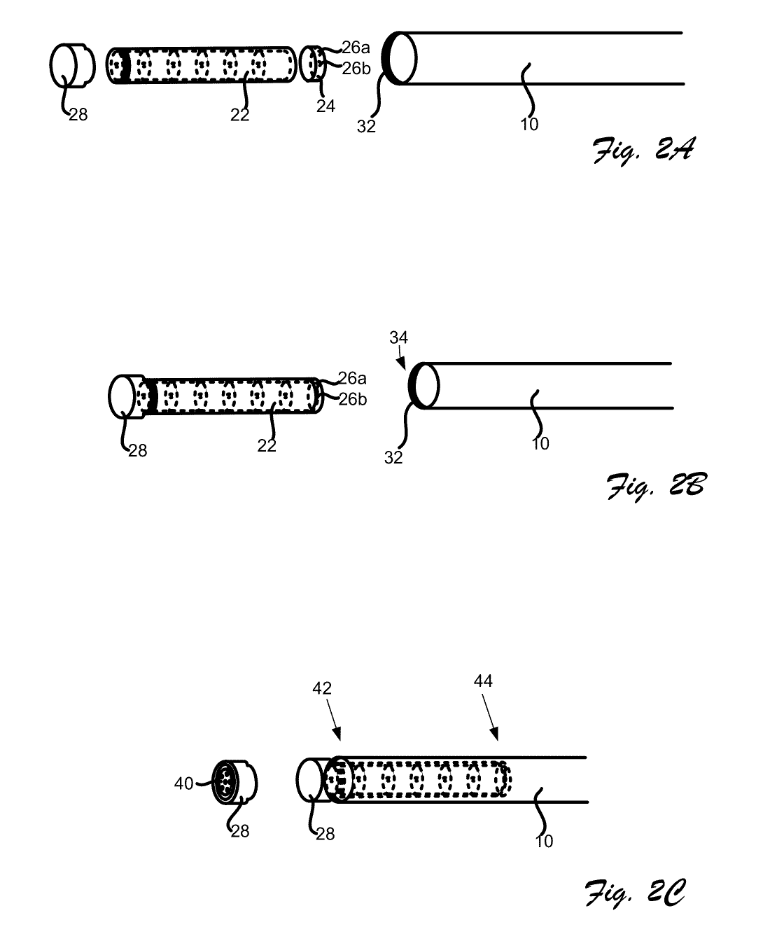

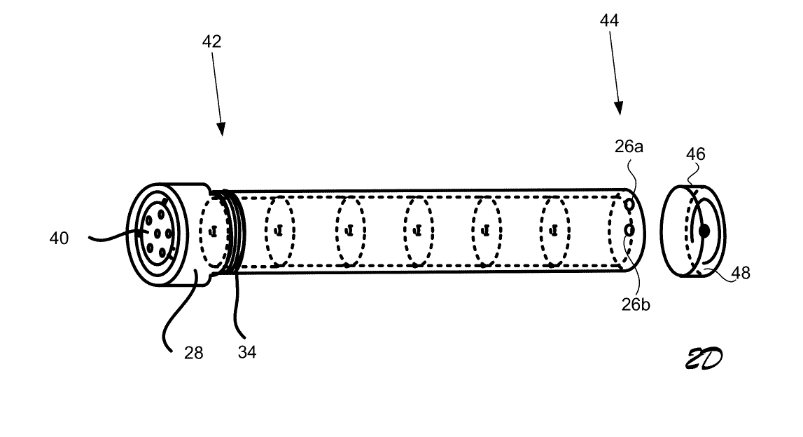

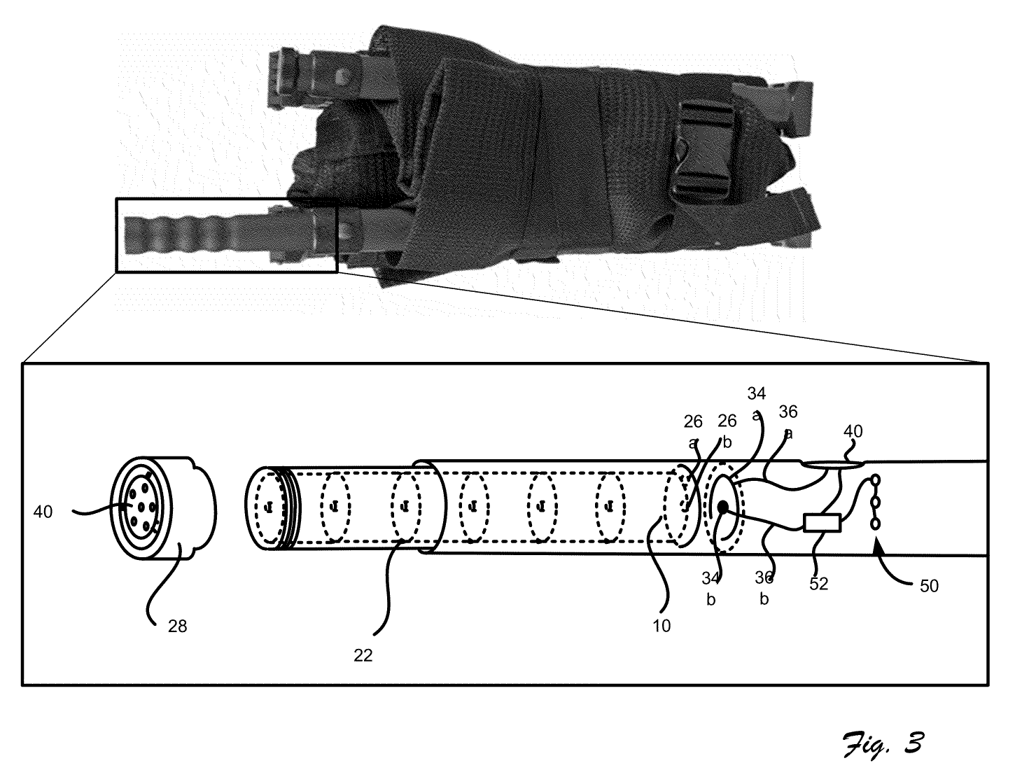

Referring to FIG. 2A, a battery sleeve 22 is shown. The battery sleeve defines a battery sleeve cavity which can receive the power cell or cells. The battery sleeve can be slotted to match rails contained within the frame section so that the sleeve can only be inserted in a specific orientation. A first end cap 24 can be removably attached to the battery sleeve at a proximal end. The first end cap can include contacts 26a and 26b. A second end cap 28 can be removably attached to the sleeve at a distal end for enclosing the battery cells in the sleeve.

Referring to FIGS. 2B and 2C, the sleeve is received into an opening 32 of a frame section. Threads 34 included in opening 32 can receive threads 34 on the sleeve securing the battery sleeve to the frame. In one embodiment, the second end cap can be attached to the frame section. Referring to FIG. 2D, first end can be attached to the proximal end 44 and include a connection plate 48. When the connection cap is attached to the proximal end, an electronic connection is made between connectors 26a and 26b. The second end cap can include a medical device connector 40 so that when the first end cap and second end cap are attached, the assembly can be used as a stand-alone power supply.

Referring to FIG. 3, the frame is shown in a collapsed position. When the sleeve is received into the frame section, electrical contacts 26a and 26b contact frame contacts 36a and 36b, respectively. Power cables 38a and 38b connect litter pole contacts 36a and 36b to medical device connector 40. Medical device connector 40 can be molded into litter pole 10, affixed or removably attached to the litter pole. In one embodiment, the connector is a BA-5590 style connector. The medical device connector can also be used to connect a recharger for recharging a rechargeable power cell or cells. In one embodiment, the power cables can be removably connected to the medical device connector so that difference connectors can be attached to the power supply.

In one embodiment, there can be indicators which can represent power levels, proper connection, power cells in use, or any combination. In one embodiment, LED indicators 50 are used. The LED indicators can have a red, yellow, or green indicator for indicating power level. The indicator can have a binary indication (on/off) indicating when there is a device connected to the power supply. Further, the indicators can provide an indication of remaining power life. The LED controller 52 can sense the battery levels, operation, and other status information, and actuate the LED indicators accordingly. The LED controller can be hardware, software or a combination.

It will be apparent to those skilled in the art that various modifications and variations can be made in the present invention without departing from the scope and spirit of the invention. It is intended that the present invention include such modifications and variations as come within the scope of the claims herein and their equivalents.

What is claimed is:

1. A portable collapsible field litter comprising:
a frame for supporting a tent and having a plurality of frame sections hingeably attached wherein said frame and said tent have a collapsed position for storage and transportation and an extended position for supporting an individual;
a cavity defined internal to one of said frame sections for receiving a power cell within said frame section;
a medical device connector attached to said frame and in electrical communications with said power cell through a plurality of contacts disposed in said cavity providing an electrical connection between said power cell an said medical device connector;
a heated wrap electrically connected to said medical device connector so that said heated wrap is heated using power delivered from said power cell, said heated wrap is disposed around the individual supported by said tent; and,
a heat loss fabric disposed around said individual and said heated wrap to reduce heat radiating away from the individual to assist in preventing hypothermia.

2. The apparatus of claim 1 wherein said medical device connector is a BA-5590 connector.

3. The apparatus of claim 1 wherein said heated wrap is a medical device taken from the group consisting of: ARCtc Cocoon BA, ARCtc HPMK BA and ARCtc ThermalWrap BA.

4. The apparatus of claim 1 including an indicator electrically connected to said power cell wherein said indicator indicates status taken from the group consisting of: power level, medical device connection, remaining power, and any combination thereof.

5. A portable collapsible field litter comprising:
a frame for supporting a tent and having a plurality of frame sections hingeably attached wherein said frame and said tent have a collapsed position for storage and transportation and an extended position for supporting an individual;
a cavity defined internal to one of said frame sections for receiving a power cell within said frame section;
a medical device connector attached to said frame and in electrical communications with said power cell through a plurality of contacts disposed in said cavity providing an electrical connection between said power cell and said medical device connector; and,
a heated wrap electrically connected to said medical device connector so that said heated wrap is heated using power delivered from said power cell, said heated wrap is disposed around the individual supported by said tent to assist in preventing hypothermia.

6. The apparatus of claim 5 wherein said heated wrap is a medical device taken from the group consisting of: ARCtc Cocoon BA, ARCtc HPMK BA and ARCtc ThermalWrap BA.

7. The apparatus of claim 1 including an indicator electrically connected to said power cell wherein said indicator indicates status taken from the group consisting of: power level, medical device connection, remaining power, and any combination thereof.

8. A portable collapsible field litter having an integrated power source comprising:
a frame for supporting a tent and having a plurality of frame sections hingeably attached wherein said frame and said tent have a collapsed position for storage and transportation and an extended position for supporting an individual;
a cavity defined internal to one of said frame sections for receiving a power cell within said frame section;
a plurality of electrical contacts disposed within said cavity so that said power cell has an electrical connection with said electrical contacts when said power cell is received in said cavity; and,
a medical device connector integrated into said frame and having an electrical connection with said electrical contacts whereas said medical device connector can provide electrical power to a medical device connected to said medical device connector.

9. The apparatus of claim 1 wherein said medical device connector is a BA-5590 connector.

10. The apparatus of claim 1 wherein said medical device is a heated wrap to assist in preventing treating hypothermia.

11. The apparatus of claim 10 wherein said heated wrap is a medical device taken from the group consisting of: ARCtc Cocoon BA, ARCtc HPMK BA and ARCtc ThermalWrap BA.

12. The apparatus of claim 8 including a power switch electrically in-line between said power cell and said medical device connector for allowing power from said power cell to be interrupted or delivered to said medical device connector when said power switch is actuated.

13. The apparatus of claim 8 including a power level indicator in electrical connection with said power cell indicating the amount of power remaining in said power cell.

14. The apparatus of claim 13 wherein said indicator is a LED light.

15. The apparatus of claim 13 wherein said indicator indicates status taken from the group consisting of: power level, medical device connection, remaining power, and any combination thereof.

16. The apparatus of claim 8 wherein said cavity is defined in a proximal end of a handle section of said frame.

17. The apparatus of claim 16 including an end cap for securing said power cell in said cavity in said handle section.

18. The apparatus of claim 8 including a sleeve for receiving said power cell whereas said sleeve containing said power cells is received in said cavity, said sleeve having an electrical connection between said power cell and said electrical contacts allowing power to be transmitted from said power cell to said electrical contacts.

19. The apparatus of claim 18 including a first end cap having a medical device connector removable attached to a proximal end of said sleeve and a second end cap having internal electrical contacts removable attached to a distal end of said sleeve.

20. The apparatus of claim 20 wherein said power cell is affixed within said cavity and rechargeable through said medical device connector.
